(19) United States
(12) Patent Application Publication
(10) Pub. No.: US 2023/0330068 A1
(43) Pub. Date: Oct. 19, 2023

(54) GEL COMPOSITION OF LULICONAZOLE FOR TREATMENT OF VAGINAL FUNGAL INFECTIONS AND ITS MANUFACTURING PROCESS

(71) Applicants: Fahad A. Al-Abbasi, Jeddah (SA); Shareefa A. AlGhamdi, Jeddah (SA); Salman Bakr Hosawi, Jeddah (SA); Amira M. Alghamdi, Jeddah (SA); Mustafa Zeyadi, Jeddah (SA); Mujaddad Ur Rehman, Khyber Pakhtunkhwa (PK); Govind Vyas, Monmouth Junction, NJ (US); Imran Kazmi, Jeddah (SA)

(72) Inventors: Fahad A. Al-Abbasi, Jeddah (SA); Shareefa A. AlGhamdi, Jeddah (SA); Salman Bakr Hosawi, Jeddah (SA); Amira M. Alghamdi, Jeddah (SA); Mustafa Zeyadi, Jeddah (SA); Mujaddad Ur Rehman, Khyber Pakhtunkhwa (PK); Govind Vyas, Monmouth Junction, NJ (US); Imran Kazmi, Jeddah (SA)

(21) Appl. No.: 18/307,390

(22) Filed: Apr. 26, 2023

(51) Int. Cl. A61K 47/38; A61K 47/14; A61K 9/06; A61K 47/34; A61K 31/4178; A61K 47/32

(52) U.S. Cl. CPC A61K 31/4178; A61K 9/06; A61K 47/14; A61K 47/32; A61K 47/34; A61K 47/38

(57) ABSTRACT

The present invention generally relates to a process for manufacturing gel composition of Luliconazole for treatment of vaginal fungal infections cooling 50% quantity of purified water at 4° C.; adding 0.10-0.30% of Carbopol 934, 5-15% of poloxamer 188 and 15-25% of poloxamer 407 slowly under constant stirring and placing at 4° C., until a clear solution is obtained; adding 0.1-0.5% HPMC K100M gradually under stirring and leaving for 24 hours at room temperature; adding 0.5-2% Luliconazole solution slowly under high speed homogenizer; adding 0-0.5% Sodium methyl parabens and 0-1% Sodium propyl parabens solution under constant stirring; adding 0.1-0.5% sodium chloride under stirring; checking and maintaining pH of the obtained solution; and preparing the obtained solution volume to 100% with purified water and stirring for 30 min for manufacturing gel composition of Luliconazole.

### FIELD OF THE INVENTION

The present disclosure relates to in-situ Gel formulation of Luliconazole for effective treatment of vaginal fungal infections and its manufacturing and preparation process thereof.

### BACKGROUND OF THE INVENTION

The vaginal cavity is an important area of female reproductive system and it acts as favorable site for drug administration because of enormous permeation area, significant vascularization and comparatively low enzymatic activity. The vaginal physiology mainly affected by age, hormonal levels, pregnancy, pH variation and concentration of micro flora. Significant changes occur in vaginal physiology with age like production of vaginal fluid, amount of vaginal discharge and change in thickness of epithelial layer. Because of these regular changes in female reproductive systems, females are more prone to fungal infections. The infections sometimes also results from variation the environmental conditions of vagina such as pH. The normal pH range of vagina is 3.5 to 5 in healthy adult women and intrinsic production of lactic acid by vaginal microbial flora is responsible for maintaining this pH. Worldwide reproductive tract infection (RTI) has become serious health concern specially developing in countries like India. As per WHO (world health organization) around 356 million cases of RTI are reported every year worldwide. Candidiasis is one of most frequently observed genital infection occurring in women of all age groups. The Candida albicans is responsible for almost 90% of the vaginal fungal infections; however in genesis of vulvovaginal candidiasis, recently it has been reported for involvement of other candida species (i.e. Candida glabrata, Candida Para psilosis) apart from Candida albicans. The manifestations of Vulvovaginal candidiasis are usually painful and uncomfortable causing intense itching, irritation, vaginal discharge and dysuria.

Generally, topical, oral and intra-vaginal routes are used for treating vaginal infections depending upon the severity. The conventional formulation like suspension, creams and solutions are unable to maintain effective drug concentration for longer period because of their short residence time at vaginal cavity. However, formulation like ointment, gel and vaginal insert provides comparatively more residence time of drug but exhibit significant difficulty in direct application to the vaginal cavity. Mucoadhesive formulation provides an interaction between mucoadhesive polymer and vaginal mucosa thereby could increase the residence time of drug at vaginal cavity, which eventually results in enhanced bioavailability. Recently in-situ gel formulations have been reported as most suitable formulation for topical administration. The in-situ gels are stimuli sensitive hydrogels, which exists as a liquid before administration and immediately changes to gel form after coming in contact with vaginal mucosa. The in-situ gel development offers number advantages over other topical formulations such as ease of administration, good Spreadability, reduce dose frequencies, increase in contact time of drug with vaginal mucosa and enhanced patients compliance.

The different Azol derivatives such as Ketoconazole, Itraconazole, Fluconazole and Luliconazole are most prescribed for the treatment of various vaginal infections. In spite of an excellent anti-fungal profile and notable literature support Luliconazole has not been much explored for treatment of vaginal fungal infections like vaginal candidiasis or Candida vulvovaginitis. It has been mostly used for treatment and management of skin, eye and nail related fungal infections such as jock itch, ringworm and fungal keratitis.

The recent findings showed that Luliconazole has potent anticandidal activity with very low minimum inhibitory concentration (MIC) (MIC range: 0.031-0.13 mg/ml), good skin safety profile, efficacy at low drug concentrations and lipophilic nature which made it an excellent drug of choice for the treatment of fungal infection. Lately the molecular docking studies of Luliconazole has reported high binding affinity and interaction activity with the cellular contents of C. albicans, subsequently, leading to cell disruption and death. Luliconazole is approved by FDA and is available in market under brand name of LUZU 1% w/w cream. The in-situ gel formulation of Luliconazole is not been reported yet.

As per WHO around 356 million cases of RTI are recorded every year worldwide. Candidiasis is major genital infection occurring in women of all age groups. The Candida albicans is responsible for almost 90% of the vaginal fungal infections. The conventional formulation like suspension, creams and solutions are unable to maintain effective drug concentration for longer period because of their short residence time at vaginal cavity. However, ointment, gel and vaginal insert formulations provides more residence time as compared to conventional formulations but causes significant difficulty in direct application to the vaginal cavity. The in-situ gel formulations offers number advantages over other topical formulations such as ease of administration, good Spreadability, reduce dose frequencies, increase in contact time of drug with vaginal mucosa and enhanced patients compliance. Luliconazole has potent anticandidal activity with very low MIC (range: 0.031-0.13 mg/ml), good skin safety profile, efficacy at low drug concentrations and lipophilic nature therefore making it an excellent drug of choice for the treatment of fungal infection. Luliconazole is approved by FDA and is available in market under brand name of LUZU 1% w/w cream. The in-situ gel formulation of Luliconazole is not been reported yet.

US2013/0123318 A1, discloses pharmaceutical compositions comprising luliconazole for effective treatment of dermatomycoses and onychomycosis. The formulation developed as liquid topical dosage form containing penetrating agents such as Transcutol P, thereby proving enhanced penetration of luliconazole through skin. The disclose invention was mainly designed for treatment of Tinea pedis or athletic foot and fungal infection of nails.

U.S. Pat. No. US 8349882 described a pharmaceutical composition for external use comprising luliconazole and propylene carbonate at concentration of 0.1 to 40% by mass. The present involves better solubilization of water insoluble luliconazole by use of solubilizing agent such aspropylege glycol, N-methyl-2-pyrrolidone in cream based topical formulation.

WO2015156219, discloses pharmaceutical composition containing a steroid and an antifungal agent. International patent application WO2011024620, discloses a pharmaceutical formulation of luliconazole with ketone and hydroxyalkyl benzene for good solubilization and suppressing stereo isomerization of luliconazole. WO2009031642, related to antifungal agent for external application 50-95% by weight of alcohol, 0.1 to 35% by weight of water and or an anionic surfactant.

WO 2014/042043 A1, discloses the embodiment containing miconazole and luliconazole as topical cream formulation. The present invention provides mean of controlling the amide impurity below 0.2% in topical cream formulation of miconazole and luliconazole. The present invention although will provide better stability but will have number of serious limitation for vaginal applications such as difficulty in administration, poor Spreadability, low residence time etc.

EP 2762139 B1, discloses the cream based formulation of luliconazole for effective treatment of mycosis. In the present invention the drug was dissolved in oil base such as medium chain triglyceride and by use of surfactants so as to provide better absorption and less irritation. However, the present formulation containing the surfactant could cause serious irritation to vagina upon application and have other limitations of cream based formulation as discussed earlier.

US20160243147A1, describe a topical anti-fungal pastes and methods that treat fungal infections of the skin, reduce the severity and duration of symptoms of fungal infections of the skin, and prevent recurrence of fungal infections. The topical pastes described in present invention are composed of an admixture of one or more antifungal agents, excipient inert powder, and a pharmaceutically acceptable topical carrier. The present invention involves use of antifungal agent such as clotrimazole, ketoconazole, miconazole, oxiconazole, econazole, luliconazole, terbinafine, nystatin, fluconazole, voriconazole, itraconazole, caspofungin, butenafine, amorolfine, ravuconazole, Posaconazole, flucytosine, sertaconazole, efinaconazole, enilconazole, saperconazole, sulconazole, terconazole, tioconazole, nikkomycin Z, anidulafungin (LY303366), pimaricin, griseofulvin, ciclopirox, haloprogin, tolnaftate, undecylenate, or any combination thereof.

The compositions and methods described herein minimize fungal resistance and maximize the number of targeted fungal strains. Additionally, the compounds and methods do not suppress the body’s immune system either locally or systemically, thus allowing for a faster restoration of normal skin flora.

WO2017203456, the present invention relates to stable topical compositions comprising Luliconazole. Further, the present invention also relates to stable topical cream formulation comprising combination of luliconazole with a corticosteroid such as betamethasone or clobetasol. The compositions are free of an aliphatic alcohol and a ketone thereby providing better stability. As the present invention is cream dosage form therefore will have number of limitation for vaginal delivery.

201941003828, this India patent discloses a topical formulation of luliconazole or its pharmaceutically acceptable salts thereof for treatment of skin fungal infection. The topical cream developed in present invention is more stable and freer from chelating agents.

201921008070, the invention related to topical pharmaceutical composition of luliconazole for treatment of fungal skin infections such tinea pedis, tinea curis, tinea corporis. The invention specifically related to topical gel formulation of luliconazole, which involve solubilization of water insoluble luliconazole by use of suitable solubilizing agent.

In spite of prominent antifungal activity of luliconazole and low MIC (range: 0.031-0.13 mg/ml) none of the prior art used Luliconazole for treatment of vaginal fungal infections such as vaginal candidiasis. All the prior arts disclose the conventional dosage forms embodiments and they have serious limitations for vaginal applications like, difficulty in administration, poor Spreadability, low retention time and poor patient compliance.

In the view of the forgoing discussion, it is clearly portrayed that there is a need to have a process to develop and evaluate in-situ gel formulation of Luliconazole with mucoadhesion property to provide effective treatment of vaginal fungal infections such as Vaginal candidiasis.

### SUMMARY OF THE INVENTION

The present disclosure seeks to provide a process for preparation in-situ gel composition of Luliconazole to provide effective treatment of vaginal fungal infections considering the efficient antifungal activity of Luliconazole and advantages of in-situ gel formulation.

In an embodiment, a gel composition of Luliconazole for treatment of vaginal fungal infections is disclosed. The composition includes 0.5-2 wt% of Luliconazole; 0.1-0.5 wt% of Hydroxypropyl methyl cellulose (HPMC-K100M); 5-15 wt% of Poloxamer 188; 15-25 wt% of Poloxamer 407; 0.10-0.30 wt% of Carbopol 934; 15-25 wt% of N-methyl-2-pyrrolidone (NMP); 0.1-0.5 wt% of Sodium chloride; 0-0.5 wt% of Sodium methyl parabens; and 0-1 wt% of Sodium propyl parabens.

In another embodiment, a process for manufacturing gel composition of Luliconazole for treatment of vaginal fungal infections is disclosed. The process includes cooling 50% quantity of purified water at 4° C.; adding 0.10-0.30% of Carbopol 934, 5-15% of poloxamer 188 and 15-25% of poloxamer 407 slowly under constant stirring and placing at 4° C., until a clear solution is obtained; adding 0.1-0.5% HPMC K100M gradually under stirring and leaving for 24 hours at room temperature; adding 0.5-2% Luliconazole solution slowly under high speed homogenizer; adding 0-0.5% Sodium methyl parabens and 0-1% Sodium propyl parabens solution under constant stirring; adding 0.1-0.5% sodium chloride under stirring; checking and maintaining pH of the obtained solution; and preparing the obtained solution volume to 100% with purified water and stirring for 30 min for manufacturing gel composition of Luliconazole.

An object of the present disclosure is to develop and evaluate of mucoadhesive In-situ gel formulation Luliconazole.

Another object of the present disclosure is to conduct irritation test with Hen’s Egg Test Chorioallantoic Membrane Test (HET-CAM).

Yet another object of the present invention is to deliver an expeditious and cost-effective gel formulation of Luliconazole with mucoadhesion property to provide effective treatment of vaginal fungal infections such as Vaginal candidiasis.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a flow chart of a process for manufacturing gel composition of Luliconazole for treatment of vaginal fungal infections in accordance with an embodiment of the present disclosure;

FIG. 2 illustrates Table 1 depicts formulation of In-situ gel of Luliconzole in accordance with an embodiment of the present disclosure;

FIG. 3 illustrates a Mucoadhesion test apparatus in accordance with an embodiment of the present disclosure; and

FIG. 4 illustrates a Syringeability test Apparatus (1: metal support; 2: plastic clamp ring; 3: Force transducer; 4: syringe in accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises...a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

In an embodiment, a gel composition of Luliconazole for treatment of vaginal fungal infections is disclosed. The composition includes 0.5-2 wt% of Luliconazole; 0.1-0.5 wt% of Hydroxypropyl methyl cellulose (HPMC-K100M); 5-15 wt% of Poloxamer 188; 15-25 wt% of Poloxamer 407; 0.10-0.30 wt% of Carbopol 934; 15-25 wt% of N-methyl-2-pyrrolidone (NMP); 0.1-0.5 wt% of Sodium chloride; 0-0.5 wt% of Sodium methyl parabens; and 0-1 wt% of Sodium propyl parabens.

In another embodiment, weight percentage of Luliconazole, Hydroxypropyl methyl cellulose (HPMC-K100M), Poloxamer 188, Poloxamer 407, Carbopol 934, N-methyl-2-pyrrolidone (NMP), Sodium chloride, Sodium methyl parabens, and Sodium propyl parabens is preferably 1%, 0.2%, 10%, 20%, 0.15%, 20%, 0.2%, 0.1%, and 0.05%, respectively.

In another embodiment, weight percentage of Hydrochloric Acid (1 N), Sodium hydroxide (1 N), and purified water is selected from 0-100%.

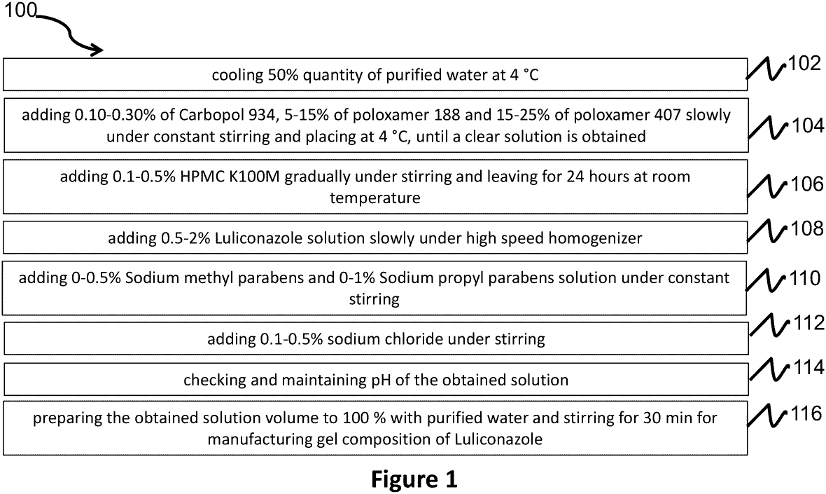

Referring to FIG. 1, a flow chart of a process for preparation/manufacturing of gel composition of Luliconazole for treatment of vaginal fungal infections is illustrated in accordance with an embodiment of the present disclosure. At step 102, process 100 includes cooling 50% quantity of purified water at 4° C.

At step 104, process 100 includes adding 0.10-0.30% of Carbopol 934, 5-15% of poloxamer 188 and 15-25% of poloxamer 407 slowly under constant stirring and placing at 4° C., until a clear solution is obtained.

At step 106, process 100 includes adding 0.1-0.5% HPMC K100M gradually under stirring and leaving for 24 hours at room temperature.

At step 108, process 100 includes adding 0.5-2% Luliconazole solution slowly under high speed homogenizer.

At step 110, process 100 includes adding 0-0.5% Sodium methyl parabens and 0-1% Sodium propyl parabens solution under constant stirring.

At step 112, process 100 includes adding 0.1-0.5% sodium chloride under stirring.

At step 114, process 100 includes checking and maintaining pH of the obtained solution.

At step 116, process 100 includes preparing the obtained solution volume to 100% with purified water and stirring for 30 min for manufacturing gel composition of Luliconazole.

In one embodiment, preferably 1% Luliconazole is separately dissolved in 15-25% NMP, preferably 20% for preparing Luliconazole solution.

In one embodiment, the Sodium methyl parabens and Sodium propyl parabens solution is prepared upon dissolving 0.1% Sodium methyl parabens and 0.05% Sodium propyl parabens separately in 10% of purified water.

In one embodiment, the pH of solution is maintained upon adding HCL (1 N) or NaoH (1 N) to reach pH 3-5, if required.

In one embodiment, the 50% quantity of purified water is cooled at 4° C., followed by adding preferably 0.15% Carbopol 934, 10% poloxamer 188 and 20% poloxamer 407 slowly under constant stirring using a magnetic stirrer.

In one embodiment, preferably 0.2% HPMC K100M is added gradually under stirring to the obtained clear solution and left for 24 hours at room temperature.

In one embodiment, preferably 0.2% sodium chloride is added after adding Sodium methyl parabens and Sodium propyl parabens solution under stirring.

### Manufacturing/Preparation Process

Step 1: The 50% quantity of purified water is cooled at 4° C., Carbopol 934, poloxamer 188 and poloxamer 407 are added slow under constant stirring.

Step 2: Step 1 gel is placed at 4° C., until a clear solution is obtained.

Step 3: Now HPMC K100M is added gradually under stirring to step 2 gel and left for 24 hours at room temperature.

Step 4: Luliconazole is dissolved separately in NMP.

Step 5: Step 4 Luliconazole solution is slowly added to step 3 solution under high speed homogenizer.

Step 6: Sodium methyl parabens and Sodium propyl parabens is dissolved separately in 10% of purified water.

Step 7: Step 6 solution is added in to step 5 under constant stirring.

Step 8: Now sodium chloride is added under stirring to step 7 solution.

Step 9. Check the pH of solution and added HCL (1 N) or NaoH (1 N) to reach pH 3-5, if required

Step 10: Now make up the volume to 100 % with purified water and stir for 30 min.

### Evaluation of In-Situ Gel Formulation of Luliconazole

Drug-excipients compatibility Study: Compatibility studies are carried out to find out whether the polymeric excipients used in formulation are compatible with drug throughout shelf life. These studies have been performed by using differential scanning calorimetry, using aluminum hermetic pans with pierced lid over range of 30° C. to 300° C., at a scan rate of 10° C./min with nitrogen at the flow of 50 mL/min as a purge gas. The Fourier transform infrared spectroscopy using KBr pellets are conducted at a scan range of 450-4000 cm⁻¹.

Determination of pH: To investigate the compatibility of the gel bases for vaginal application, their pH values are determined by a pH meter (Eutech pH 2700) at room temperature (n = 5). The equilibration time for pH measurement is taken 1 minutes.

Determination of viscosity: The viscosity of all formulated batches of the in-situ gel is measured using Brookfield Digital Viscometer (DV-E model). The tests are performed in triplicate.

Determination Gelation Temperature and Time: A 5 ml sample of the in-situ gel formulation is transferred to a test tube, submerged in a thermostat water bath. The temperature of the water bath is raised with an increment of 1° C. per 30 seconds and kept for 5 min to equilibrate at every new set of temperatures. After that, the sample is inspected for gelation by slanting the test tube and it is believed to take place when the meniscus would no longer move. The temperature at which meniscus no longer moves is recorded as Gelation temperature and time taken to reach Gelation temperature from start of study is recorded as Gelation time. Each sample is measured in triplicate.

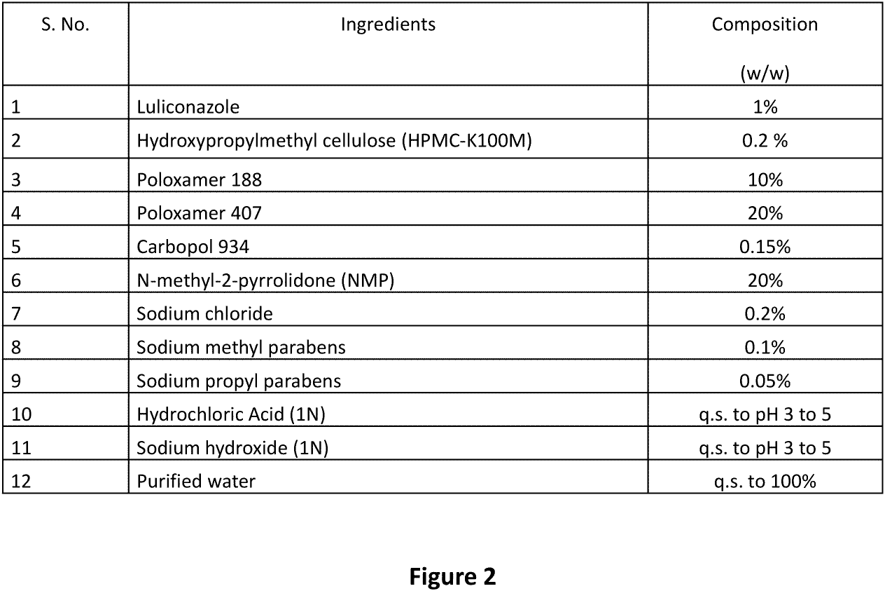

FIG. 2 illustrates Table 1 depicts formulation of In-situ gel of Luliconzole in accordance with an embodiment of the present disclosure.

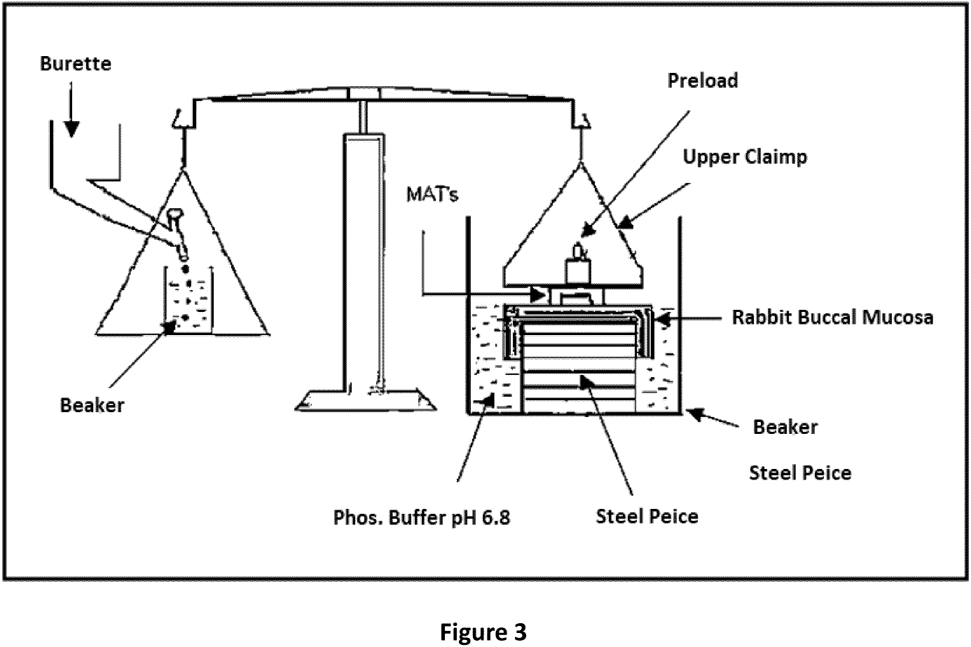

FIG. 3 illustrates a Mucoadhesion test apparatus in accordance with an embodiment of the present disclosure. The mucoadhesive force is a measure of a force necessary to separate the formulation from vaginal mucosal membrane. Mucoadhesive property is determined using modified physical balance. Sheep buccal mucosa is used as biological membrane, which is fixed under one pan of the balance with the help of cyanoacrylate glue and is hydrated with 100 µL of phosphate buffer pH 4.5 maintained at 37± 1° C. Accurately weighed amount of 1 g of luliconazole in-situ gel formulation is stuck to the inverted beaker (250 mL) using glue and the height of the balance is adjusted to accommodate a glass container below the pan where membrane is glued. A preload of 20 g is applied in order to allow the formation of mucoadhesive joints. After a 3 min rest period, the preload is removed and gradually the weight is added to the other pan until the gel is detached from the mucosal surface. The total weight required for the complete detachment of the gel is recorded.

### Spreadability Test

To determine the Spreadability of the developed formulation, approximately 1 g of in-situ gel is placed at the center of the glass plate (20 cm × 20 cm). This glass plate is covered with another glass plate of the same size. Next, the weight of 1000 g is carefully applied on the upper side of the plate; as a result the gel is spread out in between the plates. After one minute the weight is removed and the diameter of the spread area (cm) is measured. This determination is carried out in triplicate.

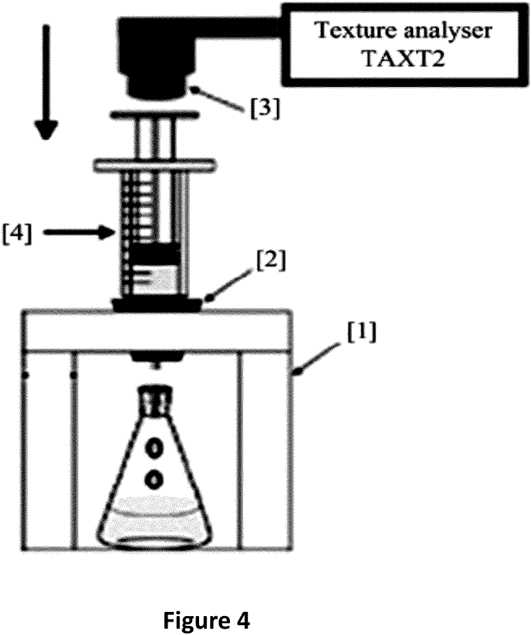

FIG. 4 illustrates a Syringeability test Apparatus (1: metal support; 2: plastic clamp ring; 3: Force transducer; 4: syringe in accordance with an embodiment of the present disclosure. The syringeability of the formulations is examined using a software controlled penetrometer in compression mode. A filled 2 mL syringe is held in place with a clamp and the upper probe of the texture analyzer moved downwards until it came in contact with the syringe barrel base. A constant force of 0.5 N is applied to the base and the work required to expel the contents for a barrel length of 30 mm is measured. The tests are conducted at room temperature and each experiment is carried out three times.

Drug Content: Drug content of in-situ gel formulation is determined by dissolving 1 ml of formulation in methanol. After suitable dilution, absorbance maximum is recorded. The concentration of the drug is determined spectrophotometrically at a λmax of 299 nm using the methanol as a blank. Drug content is determined using the slope of the standard curve.

In-vitro drug release study: Franz diffusion cell apparatus (Electrolab, India; Model EDC07) is used for an in-vitro drug release study with water-jacketed receptor chamber (20 ml) and a donor chamber maintained at 37 ± 1° C. The receptor chamber is containing phosphate buffer pH 4.0 solutions is constantly stirred by a electromagnetic stirrer. Both the chambers separated by cellulose membrane (Filter paper Whatman 41, 20-25 µm, Whatman GmbH, Dassel, Germany) and each vaginal in-situ gelling formulation spread on the circular portion of the membrane. At specified time intervals, 1 mL of the solutions are removed from the acceptor phase at each sampling time for up to 8 hrs. The aliquots are replaced with an equal volume of the freshly prepared release medium kept at the same temperature and amount of Luliconazole release is measured at 299 nm against a blank with UV spectrophotometer (JASCO, UV-VIS spectrophotometer, Japan).

Irritation test with Hen’s Egg Test Chorioallantoic Membrane Test (HET-CAM): in the present study, fertilized hen’s eggs are taken from a poultry farmhouse. Three eggs are selected for the developed in-situ gel formulation to study in triplicate, weighing between 50-55 g. These (special pathogen-free) eggs are incubated at 37° C. ± 0.5° C. in a standard cell culture incubator up to 3 days. On the 3rd day, 3 ml of egg albumin is removed by using sterile technique through a hole made by pointed pole and then the hole sealed by 70% alcohol sterilized paraffin with the help of the heated spatula. The eggs are kept in the equatorial position for the development of CAM. The eggs are candled on the 5th day of incubation and thereafter non-viable embryos are removed. On the 10th day, in-situ gelling formulation is instilled directly onto CAM surface with a pipette and observed within a specific time limit. The membrane is examined for vascular damage and the time required for injury to take place is recorded. A 0.9% Sodium Chloride (NaCl) solution is utilized as a control as it is stated to be experimentally non-irritant.

The scores are recorded according to the scoring schemes mentioned as below:
Score 0 indicates no visible hemorrhage (non-irritant);
Score 1 indicates just visible membrane discoloration (mild irritant);
Score 2 indicates structures are covered partially due to membrane discoloration and hemorrhage (moderately irritant);
Score 3 indicates structures are covered totally due to membrane discoloration and hemorrhage (severe irritant).

Stability studies: Stability studies are carried out on developed in-situ gel formulation as per ICH guideline at accelerate condition (40° C./75% RH) and at log term condition (25° C./60% RH). After specified interval formulation is checked for all evaluation parameters.

The developed invention has the following advantages over the existing formulations.
The present invention provides increase residence time of drug at target site thereby increasing the bioavailability of the drug.
The present invention provides ease of drug administration at the vaginal cavity.
The present invention exhibit excellent spreadability, hence drug reached to all area of infection.
The present invention provides longer drug action due to increased contact time with vaginal mucosa thereby reducing the dose frequency.
The present invention will exhibit reduced side effects due to its longer duration of action and decreased frequency of administration.
The present invention significantly increases the patient compliance due ease of application, reduced dose frequency and long duration of action.
The present invention will provide better and effective treatment of various vaginal fungal infections.

Present invention also has some challenges as below
The present invention requires use of a special in-situ gelling agent for formulation. Hence, availability of this agent is critical factor.
The characterization of in-situ gelling agent is very essential part as it could results in batch-to-batch variation.
The development of present invention is a complex process and it involves use of sophisticated equipments like high-speed homogenizer.
The present invention is more susceptible to stability problem due to chemical degradation. Hence, effective and strong packing material should to be used to protect it from harmful effect of environment.
The present invention remains in liquid form but when administer to vaginal cavity immediately converts to gel by the stimuli of body temperature. Hence, body temperature is a critical factor to be maintained for success of the present invention.

The developed process 100 provides ease of administration to target site, increases residence time hence providing higher bioavailability, provides longer duration of action, exhibits excellent Spreadability thereby covering all area of infection at target site, reduces frequency of drug administration, reduces adverse effect of drug due to long duration of action, increases patient compliance, and better and efficient treatment of vaginal fungal infections thereby reduced cost of treatment.

The composition is used in the field of Medicine for treatment of various vaginal fungal infection and process is used in pharmaceutical industries for manufacturing the product.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A gel composition of Luliconazole for treatment of vaginal fungal infections, the composition comprises:
0.5-2 wt% of Luliconazole;
0.1-0.5 wt% of Hydroxypropyl methyl cellulose (HPMC-K100M);
5-15 wt% of Poloxamer 188;
15-25 wt% of Poloxamer 407;
0.10-0.30 wt% of Carbopol 934;
15-25 wt% of N-methyl-2-pyrrolidone (NMP);
0.1-0.5 wt% of Sodium chloride;
0-0.5 wt% of Sodium methyl parabens; and
0-1 wt% of Sodium propyl parabens.

2. The composition of claim 1, wherein weight percentage of Luliconazole, Hydroxypropyl methyl cellulose (HPMC-K100M), Poloxamer 188, Poloxamer 407, Carbopol 934, N-methyl-2-pyrrolidone (NMP), Sodium chloride, Sodium methyl parabens, and Sodium propyl parabens is preferably 1%, 0.2%, 10%, 20%, 0.15%, 20%, 0.2%, 0.1%, and 0.05%, respectively.

3. The composition of claim 1, wherein weight percentage of Hydrochloric Acid (1 N), Sodium hydroxide (1 N), and purified water is selected from 0-100%.

4. A process for preparation of gel composition of Luliconazole for treatment of vaginal fungal infections as claimed in claim 1, the process comprises:
cooling 50% quantity of purified water at 4° C.;
adding 0.10-0.30% of Carbopol 934, 5-15% of poloxamer 188 and 15-25% of poloxamer 407 slowly under constant stirring and placing at 4° C., until a clear solution is obtained;
adding 0.1-0.5% HPMC K100M gradually under stirring and leaving for 24 hours at room temperature;
adding 0.5-2% Luliconazole solution slowly under high speed homogenizer;
adding 0-0.5% Sodium methyl parabens and 0-1% Sodium propyl parabens solution under constant stirring;
adding 0.1-0.5% sodium chloride under stirring;
checking and maintaining pH of the obtained solution; and
preparing the obtained solution volume to 100% with purified water and stirring for 30 min for manufacturing gel composition of Luliconazole.

5. The process of claim 4, wherein preferably 1% Luliconazole is separately dissolved in 15-25% NMP, preferably 20% for preparing Luliconazole solution.

6. The process of claim 4, wherein the Sodium methyl parabens and Sodium propyl parabens solution is prepared upon dissolving 0.1% Sodium methyl parabens and 0.05% Sodium propyl parabens separately in 10% of purified water.

7. The process of claim 4, wherein the pH of solution is maintained upon adding HCL (1N) or NaoH (1N) to reach pH 3-5, if required.

8. The process of claim 4, wherein the 50% quantity of purified water is cooled at 4° C., followed by adding preferably 0.15% Carbopol 934, 10% poloxamer 188 and 20% poloxamer 407 slowly under constant stirring using a magnetic stirrer.

9. The process of claim 4, wherein preferably 0.2% HPMC K100M is added gradually under stirring to the obtained clear solution and left for 24 hours at room temperature.

10. The process of claim 4, wherein preferably 0.2% sodium chloride is added after adding Sodium methyl parabens and Sodium propyl parabens solution under stirring.
